(19) United States
(12) Patent Application Publication
(10) Pub. No.: US 2010/0062437 A1
(43) Pub. Date: Mar. 11, 2010

(54) PARALLEL PROFILLING OF SIGNALING PATHWAYS

(76) Inventor: David S. Lawrence, Chapel Hill, NC (US)

Correspondence Address: AMSTER, ROTHSTEIN & EBENSTEIN LLP, 90 PARK AVENUE, NEW YORK, NY, 10016, US

(21) Appl. No.: 12/449,121

(22) PCT Filed: Jan. 16, 2008
(86) PCT No.: PCT/US2008/000613

§ 371 Date: Oct. 29, 2009

Related U.S. Application Data
(60) Provisional application No. 60/898,878, filed on Feb. 1, 2007.

(51) Int. Cl. C12Q 1/68

(52) U.S. Cl. 435/6

(57) ABSTRACT

Methods for identifying signaling pathways that regulate the expression of a test gene are provided.

### CROSS-REFERENCE TO RELATED APPLICATION

This application claims the benefit of U.S. Provisional Application No. 60/898,878, filed Feb. 1, 2007, the content of which is incorporated by reference.

### STATEMENT OF GOVERNMENT SUPPORT

The invention disclosed herein was made with U.S. Government support under grant numbers NS044429 and R33CA083208 from the National Institutes of Health. Accordingly, the U.S. Government has certain rights in this invention.

### FIELD OF THE INVENTION

The present invention generally relates to signaling pathways. More specifically, the invention is directed to methods of identifying signaling pathways that regulate expression of genes.

### BACKGROUND OF THE INVENTION

The genome-wide response of cells or tissues to environmental change is readily assessed via gene microarrays. However, in many instances, the signaling pathways responsible for the up- or down-regulation of a particular gene or group of genes are unknown. Thus, there is a need for methods for identifying signaling pathways that regulate genes of interest. The present invention addresses that need.

### SUMMARY OF THE INVENTION

The present invention is directed to a method for identifying signaling pathways that regulate the expression of a test gene. The method comprises

(a) combining each of n cell cultures with each of n compounds, where a different compound is combined with each cell culture, wherein the n cell cultures are capable of expressing the test gene, and wherein signaling pathways affected by each of the compounds are known;

(b) measuring expression of the test gene in each of the cell cultures to identify compounds (“active compounds”) that affect expression of the test gene in the cell culture;

(c) combining cell cultures with an active compound identified in step (b) and to at least a portion of the n compounds, where a different compound is combined with each cell culture;

(d) measuring expression of the test gene in each of the cell cultures of step (c) to identify compounds that negate the effect that the active compound has on expression of the test gene (“negating compounds”); and

(e) identifying the signaling pathways that are affected by a negating compound (“test gene pathways”), wherein the test gene pathways are signaling pathways that regulate the expression of the test gene.

### BRIEF DESCRIPTION OF THE DRAWINGS

FIG. 1 is a micrograph showing the visualization of active hSMG1 transcription sites in ouabain (10 μM)-treated DLD-1 cells via FISH. A significantly larger fraction (0.63±0.1) of ouabain-exposed cells display active hSMG1 transcription sites than untreated (0.19±0.1) cells (see FIG. 4).

FIG. 2A-2C is graphs of experimental measurements of proteolysis in DLD-1 cells under various conditions. Panel a shows the rate of proteolysis in DLD-1 cells that were (•) untreated, (□) amino acid deprived, and (∘) ouabain (10 μM) treated. Cells were incubated with [³H]Leu for 2 days followed by a chase period in which the cells were exposed to the above conditions. Panel b shows the rate of proteolysis in cells that are (•) untreated (∘) ouabain treated, in the presence (-----) or absence (-) of the lysosomal inhibitors NH₄Cl and leupeptin. Panel c shows the rate of proteolysis in (•) untreated (∘) ouabain treated, in the presence (---) or absence (-) of the macroautophagy inhibitor 3-methyladenine. The rate of total protein degradation at different times after labeling was calculated as the percentage of total radiolabeled protein transformed into soluble amino acids. Values are the mean±S.E. of three different wells.

FIG. 3A-3C is graphs of experimental measurements of proteolysis or protein synthesis in HEK293 cells under various conditions. Panel a shows the rate of proteolysis in HEK293 cells that were (•) untreated or (∘) exposed to siRNA against hSMG1 in the presence (-) or absence (----) of amino acids. Panel b shows the rate of proteolysis in cells (⊚) untreated or (∘) exposed to siRNA against hSMG1 (----) in the presence or absence (-) of the lysosomal inhibitors NH₄Cl and leupeptin. Panel c shows the rate of protein synthesis in HEK293 cells that were (•) untreated or (∘) exposed to siRNA against hSMG1. Protein synthesis is expressed as the amount of radioactivity incorporated (in dpm) in the acid precipitable fraction (protein) at each time. Values are the mean±S.E. of two different experiments with triplicate wells.

FIG. 4 is a micrograph showing the visualization of active hSMG1 transcription sites in untreated DLD-1 cells via FISH. A significantly smaller fraction (0.19±0.1) of untreated cells display active hSMG1 transcription than ouabain-treated (0.63±0.1) cells.

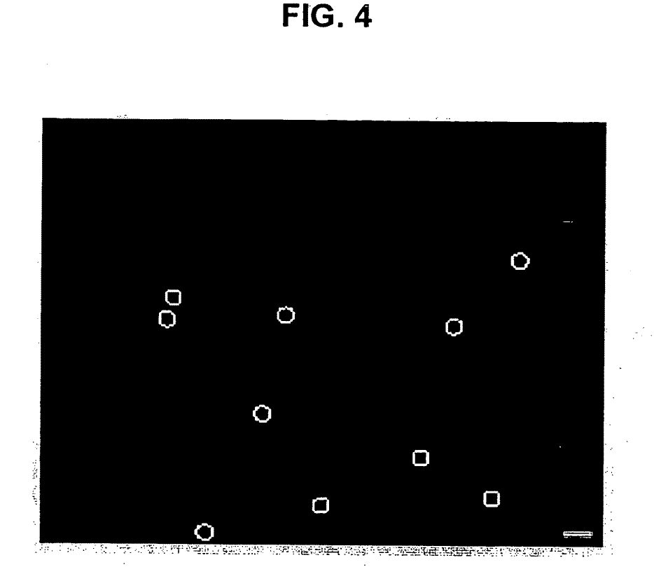

FIG. 5 is photographs showing hSMG1 protein quantitation via Western blot for untreated (control) cells (lane 1), 1 μM ouabain-treated cells for 24 hr (lane 2), and 10 μM ouabain-treated cells for 24 hr (lane 3).

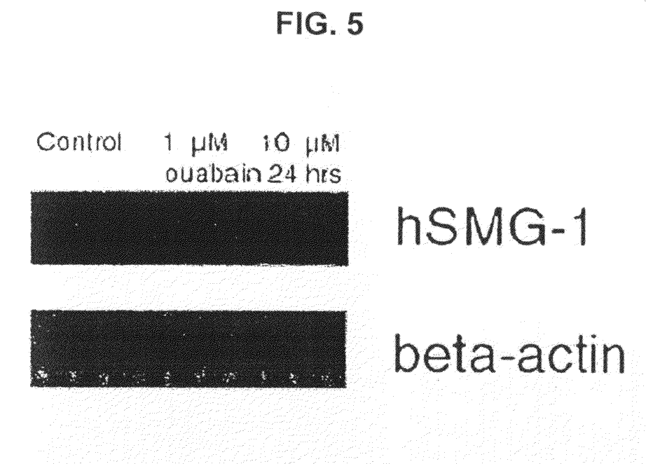

FIG. 6 is photographs of western blots and graphs showing hSMG1 protein knockdown via RNAi in HEK293 and DLD-1 cells. The results are from two western blot experiments (the first 3 lanes and the last eight lanes). Sequences of 5 siRNA duplex: si-1: gugaagauguucccuaugauu; si-2: gagguuagcugeggaaagauu; si-3: ggucagacauccaccagaauu; si-4: uaacuuggcucagcuguauuu; si-5: ccaggacacgaggaaacug; control siRNA: luciferase.

FIG. 7 is photographs of western blots and a graph showing expression changes of hSMG-1 protein in HEK293 cells. siRNA-3 (ggucagacauccaccagaauu) was used in the knockdown experiment. Amino acid starvation commenced 48 hr after siRNA transfection and lasted for 24 hours. The protein inhibitors (pepstatin and leupeptin) were added 6 hr after amino acid starvation. Lane 1 [si (−); aa (+); inhibitors (−)]; Lane 2 [si (−); aa (+); inhibitors (+)]; Lane 3 [si (−); aa (−); inhibitors (−)]; Lane 4 [si (−); aa (−); inhibitors (+)]; Lane 5 [si (+); aa (+); inhibitors (−)]; Lane 6 [si (+); aa (+); inhibitors (+)]; Lane 7 [si (+); an (−); inhibitors (−)]; Lane 8 [si (+); aa (−); inhibitors (+)].

### DETAILED DESCRIPTION OF THE INVENTION

The present invention is directed to a method for rapidly identifying signaling pathways that regulate the expression of a gene of interest (“test gene”). The method involves the combining of cells that can express the test gene with a library of compounds, where the signaling pathways that are affected by each of the test compounds are known. The compounds from the library that affect the expression of the test gene (“active compounds”) are identified from this screening. The cells are then screened with the library of compounds, along with one of the active compounds. Compounds from the library that negate the effect of the active compound on expression of the test gene (“negating compounds”) are identified from this gene. The signaling pathways that are affected by the negating compounds are then identified. These signaling pathways are those that regulate the expression of the test gene.

Thus, the invention is directed to a method for identifying signaling pathways that regulate the expression of a test gene. In the first step of the method, each of n cell cultures is combined with each of n compounds, where a different compound is combined with each cell culture. In this step, the n cell cultures are capable of expressing the test gene, and signaling pathways affected by each of the compounds are known. Next, expression of the test gene in each of the cell cultures is measured to identify compounds (“active compounds”) that affect expression of the test gene in the cell culture. The cell cultures with an active compound as identified in the measuring step are then combined to at least a portion of the n compounds, where a different compound is combined with each cell culture. Then, expression of the test gene in each of these cell cultures are measured to identify compounds that negate the effect that the active compound has on expression of the test gene (“negating compounds”). Finally, the signaling pathways that are affected by a negating compounds are identified (“test gene pathways”). These test gene pathways are signaling pathways that regulate the expression of the test gene.

The method is useful for identifying signaling pathways that regulate the expression of any test gene. Nonlimiting examples of useful test genes that can be analyzed by the method include disease genes (i.e., a gene that is associated with a disease), immunity genes, cytokine genes, or transcription factor genes. Preferably, the test gene encodes an enzyme. Where the test gene encodes an enzyme, preferred enzymes include phosphatases or kinases, due to the frequent utilization of such enzymes in signaling pathways. More preferably, the enzyme is a protein kinase, for example a member of the phosphoinositide 3-kinase-related protein kinase (PIKK) family (see Example). Other preferred test genes encode signaling molecules.

The test gene can be in any desired form that can be expressed from the cells, e.g., a splice variant or in a mutant form that is associated with disease. The gene can also be under any useful genetic control, for example under particular expression conditions. Nonlimiting examples include disease genes under conditions where it causes disease, such as where overexpression or underexpression causes disease, e.g., where a promoter or another control element is altered from wild type to cause disease, or the gene comprises a mutation that causes expression of a mutant protein that causes disease.

The cells used in the invention method are not narrowly limited to any particular type of cell, provided the cell can express the test gene. The cells can be prokaryotic cells, plant cells, or, preferably mammalian cells, such as human cells. Preferred human cells include cells that can be easily cultured to express the test gene, for example HeLa cells.

The cells can express the test gene naturally or the cell can be transfected with the test gene in a form that allows the transgene expression.

Expression of the test gene can be determined by any known method. For example, expression of the test gene can be determined by measuring mRNA levels of the test gene, e.g., by RT-PCR. Alternatively, the test gene can be expressed as an assayable peptide upon expression of the test gene, since such peptides are easily detected, e.g., with ELISA. Examples of such assayable peptides are antigens, enzymes, and fluorescent proteins. The peptides can be expressed concomitantly with the test gene of interest by, e.g., transfecting the cell with the test gene operably linked (for example as a fusion protein) to the assayable peptide.

In the invention method, the active compound could cause an increase or a decrease in expression of the test gene.

The present method is not narrowly limited to the use of any type of compound, provided that signaling pathways affected by each compound is known. For example, the compound can be a protein (including oligopeptides and polypeptides), a lipid, a nucleic acid, a carbohydrate, or any combination thereof. Preferably, the compounds are different organic compound less than about 2000 Da. Most preferably, the compounds are FDA-approved drugs or other reagents with known biological targets.

The steps of combining the cell cultures with an active compound and subsequent steps of the invention method are preferably performed with more than one active compound (if identified), e.g., a second active compound. Most preferably, these steps are performed with each active compound.

Preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the examples.

### Example

### Parallel Profiling of Signaling Pathways: hSMG1 Influences the Pace of Protein Metabolism

The human genome contains approximately 30,000 genes. However, the signaling pathways that control the expression of many of these genes remain elusive, as do the roles played by the proteins encoded by these genes. A screening strategy is described that uses a library of drugs approved for human use, to examine these issues for the phosphoinositide 3-kinase-related protein kinase (NICK) hSMG1. The results from this screen, in combination with a series of knockdown experiments, link hSMG1 to the biochemical pathways controlling the global rates of protein degradation and synthesis.

FDA-approved drugs are the most thoroughly understood of all biologically active agents. Their safety profile in humans has been carefully examined, their mechanism of action elucidated, and (in the majority of cases) their biochemical targets identified. In short, this family of biological modifiers represents a unique resource (Root et al., 2003; Savchuk et al., 2004) that could be used to globally probe cellular biochemical pathways in order to assess their influence on gene expression. A library of FDA-approved drugs and other low molecular weight compounds with well-defined biochemical targets was assembled (Xi et al., 2004). This library was employed to identify biochemical effectors that influence the expression of SMG1. SMG1 is a protein kinase implicated in the degradation of nonsense-containing mRNA and as a sensor of damaged DNA (Yamashita et al., 2001; Abraham, 2004b). Furthermore, it has been suggested that agents which augment human (h)SMG1 activity may be therapeutically useful for the treatment of ataxia telangiectasia (i.e. where high hSMG1 levels might biochemically compensate for the loss of the ATM protein kinase in these patients) (Abraham, 2004b).

The compounds in the library were acemetacin, acetamido(4-)phenol, acetohexamide, acetylsalicyclic acid, acycloguanosine, warfarin, allopurinol, alverine, althiazide, amantadine, amiloride, aminoglutethimide, amoxapine, aniracetarn, antazoline, antipyrine, thioguanine, apomorphine hydrochloride, astemizole, atenolol, atropine, azlocillin, bendroflumethiazide, benzthiazide, bepridil, betahistine, betamethasone, bezafibrate, bretylium tosylate, budesonide, bumetanide, bupropion, busulfan, biphenylacetic acid, canrenoic acid, captopril, cefaclor, cefadroxil, chloramphenicol, chlormezanone, chloroquine, chlorpheniramine, ciclopirox olamine, cinnarizine, cinoxacin, cis-platinum(II)diamine, clemastine, clotrimazole, colchicines, cyproheptadine, cyclizine, chlorpropamide, clomipramine, clozapine, debrisoquine sulfate, deprenyl hydrochloride, floxuridine, diazoxide, diclofenac, dicyclomine, diethylstilbestrol, diflunisal, digitoxin, digoxin, dihydroergotamine, dimenhydrinate, dimethyl(1,1)biguanide, diphenhydramine, DOPA, disopyramide, phenyloin, doxepine, doxylamine, domperidone, econazole, erythromycin, eserine, ethacrynic acid, ethambutol, ethyl p-aminobenzoate, etoposide, mestranol, estradiol, famotidine, fenbufen, fenofibrate, fenoprofen, fenoterol, flavoxate, fluconazole, flunisolide, fluoro(5-)uracil, flupenthixol-cis, fluphenazine, flurbiprofen, flutamide, furosemide, flucytosine, gemfibrozil, guanethidine, haloperidol, hexamethylenetetramine, hydralazine, hydrochlorothiazide, hydrocortisone, hydroflumethiazide, hydroxyzine, ibuprofen, imipramine, indapamide, indomethacin, ipratropium bromide, isonicotinic acid hydrazide, isosorbide dinitrate, kanamycin sulfate, ketoprofen, ketotifen, ketoconazole, isoproterenol-(−), labetalol, lidocaine, lithium carbonate, loperamide, magnesium sulfate, mannitol-(D), maprotiline, meclofenamic acid, mefenamic acid, acetoxy(17 alpha)-6-methyl-4,6-pregnadiene-3,20-dione, metaproterenol, metoclopramide, metolazone, metoprolol, metricane, melphalan, mercapto(6-)purine, methyl (6alpha)-17-alpha-hydroxyprogesterone, medrol, miconazole, minoxidil, mitoxantrone, nabumetone, nadolol, naphazoline hydrochloride, naproxen, nefopam, neomycin sulfate, (−)-nicotine, nimodipine, nitrendipine, nitrofurantoin, nortriptyline, oxybutynin chloride, oxymetazoline hydrochloride, oxytetracycline, penicillin G, phenelzine, pheniramine, phenoxymethyl penicillinic acid, phentolamine hydrochloride, phenyl(trans-2)cyclopropylamine, phenylbutazone, phenylephrine hydrochloride-(L), piracetam, prazosin, prednisolone, primaquine, probenecid, procainamide, propantheline bromide, propyl(2-) pentanoic acid sodium salt, propyl(3-)xanthine, promethazine, protriptyline, pyrazinamide, pyrilamine, pyrimethamine, quinidine, quinine, ranitidine, salbutamol, salicylic acid sodium salt, scopolamine, sodium nitroprusside, sotalol, spironolactone, sulconazole, sulfacetamide, sulfamerazine, sulfanilamide, sulfamethoxazole, sulfasalazine, sulfathiazole, sulindac, salicylic acid salicylate, sulfadiazine sodium salt, terazosin, terbutaline, tetrahydrozoline, thalidomide, thioridazine, ticlopidine, timolol, tinidazole, tolazamide, tolbutamide, tolfenamic acid, tolmetin, trazodone, triamcinolone, triamterene, trichlormethiazide, trichloro(2,2,2)-1-hydroxyethylphosphonic acid dimethyl ester, trihexylphenidyl, trimeprazine, trimethobenzamide, verapamil, xylometazoline, zopiclone, dextromethorphan, primidone, caffeine, furazolidone, dextrorphane tartrate, phenylacetylurea, L-thyroxine, glipzide, chlorpromazine, amphetamine(D) sulfate, butalbital, cannabidiol, cannabinol, clonazepam, codeine, desmethyldiazepam, diazepam, fenluramine hydrochloride, flunitrazepam, hydromorphone hydrochloride, meperidine hydrochloride, meprobamate, mescaline hydrochloride, methadone hydrochloride, methamphetamine(+) hydrochloride, nalorphine hydrochloride, pentobarbital, Phenobarbital, temazepam, secobarbitol, tetrahydrocannabinol (delta 8), tetrahydrocannabinol (delta 9), amylobarbitone, vincristine, daunorubicin, galanthamine, vinblastine, mitomycin C, actinomycin D, epirubicin, methylsergide, oxycodeine hydrochloride, aminophylline, azathioprine, capreomycin, carmustine, chlortetracycline, cycloserine-(D), cycloserine-(L), demeclocycline, diphenylpyraline, dipyridamole, etidronic acid disodium salt, griseofulvin, guanabenz acetate, L-3,4-dihydroxyphenylalanine methyl ester, amino(9)-1,2,3,4-tetrahydroacridine, lanatoside C, methotrexate, nalidixic acid, nystatin, pergolide, rifampicin, tetracycline, vitamin A, acebutolol, amikacin sulfate, amiodarone, amitriptyline, amoxicillin, amphotericin B, ampicillin trihydrate, aminone, aztreonam, beclomethasone, benzydamine, benztropine, bromo(2)-alpha-ergocryptine, buspirone, carbamazepine, beta-carbamylmethylcholine, carbenicillin, cefmetazole, cefoperazone, cefotaxime, ceftriaxone, cefuroxime, cephalothin, cephradine, chlorambucil, chlorothiazide, chlorprothixene, chlorthalidone, clofibrate, cimetidine, ciprofibrate, ciprofloxacin, clonidine, clopamide, cloxacillin, colistin, cyproterone acetate, cytosine beta-D-arabinofuranoside, dacarbazine, desipramine, dexamethasone, dicloxacillin, dihydrotachysterol, dobutamine, doxycycline, droperidol, enoxacin, epinephrine, ergocalciferol, ethionamide, flecainide, flunarizine, fusidic acid, glibenclamide, hydroxyurea, isoetharine, ivermectin, lansoprazole, lincomycin, hydroxychloroquine sulfate, mebeverine, meclizine, methoxamine hydrochloride, methyl(8-)-N-vanillyl-6-nonenamide, metronidazole, mexiletine, mianserin, diltiazem, milrinone, cyclosporin A, aclarubicin, pargyline, noscapine hydrochloride, 2-chorodeoxyadenosine, clindamycin, fluoro(2)adenine 9-beta-D-arabinofuranoside, netilmicin, doxorubicin, oxiracetam, trifluoperazine, oxprenolol, methy(alpha)1DOPA, paclitaxel, etodolac, curcumin, ketanserin tartrate, cephalexin, lovastatin, cyclophosphamide, nafcillin, (−)-Sparteine, Aloin, Berberine chloride, Caffeine, Ellagic acid, Morin, Myricetin, Naringin, (−)-Nicotine hydrogen tartrate salt, (+−)-Naringenin, Quercetin dehydrate, Rutin hydrate, (−)-Scopolamine hydrobromide trihydrate, Stigmasterol, Sanguinarine chloride hydrate, Theobromine, Aloe-emodine, Acacetin, 2-O-beta-L-Arabinopyranosyl-myo-inositol, Diosmin, (−)-Epicatechin gallate, (−)-Epigallocatechin gallate, Ergosterol, (Z)-Guggulsterone, Hesperidin, Luteolin, Capsaicin, Oleanolic acid, Polyphenon, Psoralen, Apigenin, Astaxanthin(−)-Bilobalide, Curcumin, beta-Carotene, Daidzein, Ginkgolide A, Genistein, Ginkgolide B, Kaempferol, Resveratrol, Silymarin, beta-Sitosterol, (±)-Taxifolin, moxifloxacin, ofloxacin, Z-devid-fmk, Genistein, Myxothiazol, Diphenyleneiodonium chloride, Okadaic acid-IEA, Ceramide bovine brain, TNFα, Jasplakinolide, CytochalasinB, Acridin orange, 2-APB, U-73122, 3-(4-Iodophenyl)-2-mercapto-(Z)-2-propenoic Acid, 4-(2 Åå-Aminoethyl)amino-1,8-dimethylimidazo[1,2-a]quinoxaline (BMS-345541), N-(3,5-Bis-trifluoromethylphenyl)-5-chloro-2-hydroxybenzamide (IMD-0354), 1,5-isoquinolinediol, 3,4-Dihydro-5[4-(1-piperindinyl)butoxy]-1(2H)-isoquinoline (DPQ), 2-(Morpholin-4-yl)-benzo[h]chromen-4-one (NU7026), Z-VE(OMe)ID(OMe)-FMK, Ac-AAVALLPAVLLALLAPIETD-CHO, 11R-CaN-AID, Ac-RRRRRRRRRRRGGGRMAPPRRDAMPSDA-NH2, 7-Chloro-5-(2-chlorophenyl)-1,5-dihydro-4,1-benzothiazepin-2(3H)-one, Cyclopiazonic acid, Dantrolene, Hinokitiol, ERK activation inhibitor peptide II, Calpain inhibitor II, and Tyrphostin RG 14620.

HeLa cells were separately exposed to each drug from the drug library in a multiwell plate format. After 24 h, hSMG mRNA was captured and quantified using a branched DNA assay (Collins et al., 1997). Actin mRNA levels were simultaneously measured from the same cell lysate in order to compensate for differences in viable cell number as a consequence of drug treatment. Lead drugs that up-regulated hSMG1 expression were identified and subsequently re-examined in triplicate (Table 1). Most notable of the small group of leads were digoxin and digitoxin, “cardiotonic steroids” used to treat congestive heart failure (Belz et al., 2001). Both agents bind to and inhibit the Na⁺/K⁺-ATPase (Therien and Blostein, 2000). An endogenous analogue of these compounds produced in the adrenal glands, ouabain (Schoner, 2002), likewise induced a 2-fold enhancement of hSMG1 expression levels in HeLa cells. The effect of ouabain (10 μM) was examined in other cell lines and all exhibited an hSMG1 over-expression response that range from moderate to robust (U87 glioblastoma: 1.4±0.1-fold; PC3 prostate carcinoma: 2.7±0.2-fold; HEK 293: 3.3±0.2-fold; GM02037 wild type fibroblast: 3.4±0.1-fold; DLD-1 adenocarcinoma: 4.3±0.1-fold; primary astrocyte: 5.4±0.1-fold; 786-0 renal carcinoma: 6.1±0.4-fold).

TABLE 1
Activators of hSMG1 Expression. HeLa cells were exposed to the library
of FDA-approved drugs and related agents at three different
concentrations (75 μg/mL, 7.5 μg/mL, 0.75 μg/mL). Hits
were re-examined at the indicated concentrations in triplicate.

| | Concentration | Actin | hSMG1 | hSMG1 |
|---|---|---|---|---|
| Drug | (μg/mL) | Response | Response | Expression |
| Thioguanine | 1.5 | 0.21 ± 0.01 | 0.67 ± 0.31 | 3.3 ± 0.3 |
| | 0.25 | 0.32 ± 0.01 | 0.71 ± 0.15 | 2.2 ± 0.1 |
| | 0.075 | 0.42 ± 0.01 | 0.93 ± 0.03 | 2.2 ± 0.1 |
| | 0.025 | 0.65 ± 0.06 | 0.99 ± 0.02 | 1.5 ± 0.1 |
| Digitoxin | 1.5 | 0.37 ± 0.01 | 2.57 ± 0.21 | 6.9 ± 0.2 |
| | 0.25 | 0.30 ± 0.01 | 2.52 ± 0.30 | 8.3 ± 0.3 |
| | 0.075 | 0.57 ± 0.02 | 1.53 ± 0.12 | 2.7 ± 0.1 |
| | 0.025 | 0.82 ± 0.04 | 0.92 ± 0.03 | 1.1 ± 0.1 |
| Digoxin | 1.5 | 0.37 ± 0.01 | 2.74 ± 0.20 | 7.3 ± 0.2 |
| | 0.25 | 0.34 ± 0.01 | 2.74 ± 0.20 | 8.0 ± 0.2 |
| | 0.075 | 0.66 ± 0.04 | 1.55 ± 0.16 | 2.3 ± 0.2 |
| | 0.025 | 0.88 ± 0.03 | 0.94 ± 0.10 | 1.1 ± 0.1 |
| Estradiol | 0.75 | 0.51 ± 0.01 | 2.24 ± 0.28 | 4.4 ± 0.3 |
| | 0.25 | 0.65 ± 0.04 | 1.11 ± 0.03 | 1.7 ± 0.1 |
| | 0.075 | 0.83 ± 0.02 | 1.11 ± 0.03 | 1.3 ± 0.1 |
| | 0.025 | 0.86 ± 0.01 | 0.93 ± 0.01 | 1.1 ± 0.1 |
| Haloperidol | 2.5 | 0.75 ± 0.03 | 1.07 ± 0.05 | 1.4 ± 0.1 |
| | 0.75 | 0.81 ± 0.04 | 1.20 ± 0.09 | 1.0 ± 0.1 |
| | 0.25 | 0.85 ± 0.02 | 1.03 ± 0.04 | 1.2 ± 0.1 |
| | 0.075 | 0.86 ± 0.05 | 0.82 ± 0.18 | 0.9 ± 0.2 |
| Amrinone | 7.5 | 0.37 ± 0.01 | 1.49 ± 0.13 | 4.0 ± 0.1 |
| | 1.5 | 0.54 ± 0.02 | 1.50 ± 0.16 | 2.8 ± 0.2 |
| | 0.5 | 0.66 ± 0.01 | 1.15 ± 0.07 | 1.7 ± 0.1 |
| | 0.15 | 0.82 ± 0.03 | 0.92 ± 0.02 | 1.1 ± 0.1 |

“Actin Response” refers to the ratio of actin mRNA in drug-exposed versus unexposed cells. The general decline in actin mRNA levels upon drug treatment is a consequence of cell death. This was confirmed via the CellTiter-Glo ® Luminescent Cell Viability Assay kit (Promega).
“hSMG1 Response” refers to the ratio of hSMG1 mRNA in drug-exposed versus unexposed cells.
“hSMG1 Expression” is the ratio of “hSMG1 Response” to “Actin Response”. The latter takes into account well-to-well variations in cell number.

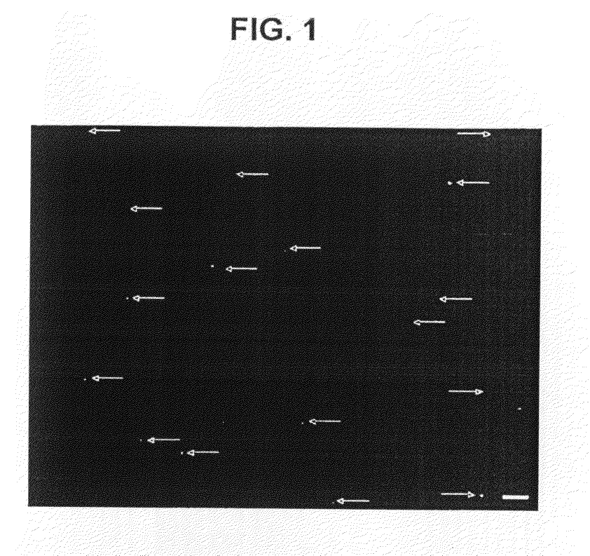

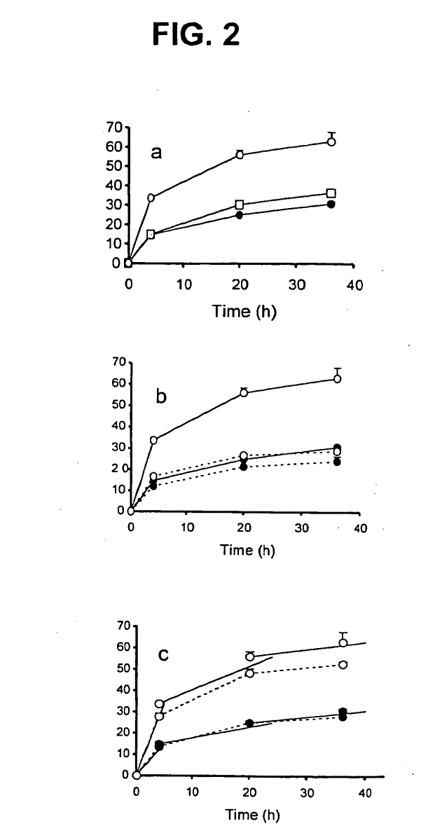

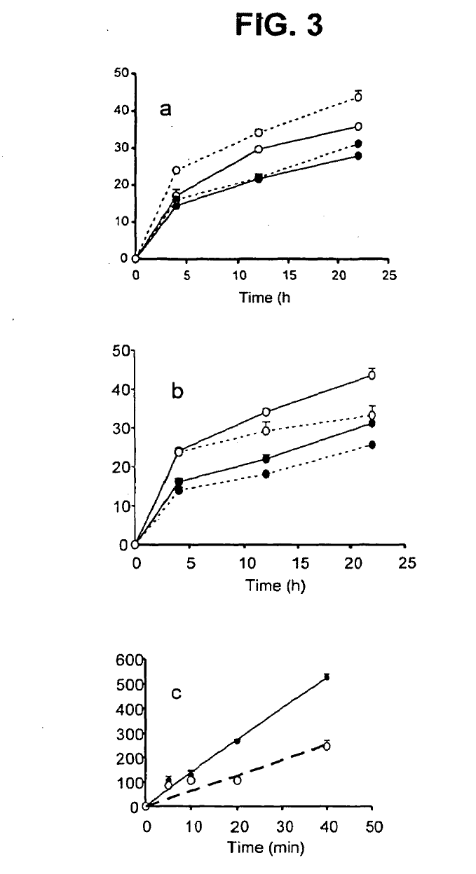

The branched chain DNA assay affords an averaged expression response from a cell population. The magnitude of this response is a function of actin mRNA levels, a baseline that furnishes relative, but not absolute, hSMG1 mRNA quantity. Since actin mRNA levels may be susceptible to influence by these drugs as well, fluorescence in situ hybridization was employed to directly visualize hSMG1 mRNA transcription sites in the DLD-1 cell line. The latter cell line was chosen because it exhibits a strong hSMG1 response to ouabain exposure. A significantly larger fraction of cells treated with ouabain (0.63±0.10) (FIG. 1) display active transcription sites than their untreated counterparts (0.19±0.01) (FIG. 3). These results demonstrate, in a fashion that is both independent of yet consistent with the branched DNA assay, that ouabain exposure promotes the activation of hSMG1 mRNA transcription. Furthermore, these enhanced levels of mRNA synthesis rate and quantity directly translate into higher proteins levels, as exemplified by the ouabain-induced 2.7-fold increase in hSMG1 protein in DLD-1 cells (FIG. 2).

hSMG1 is a member of the PIKK family, which also includes ATM, ATR, mTOR, and DNA-PK (Therien and Blostein, 2000). These large, structurally homologous, proteins have been implicated as participants in the cellular response to various forms of stress. Although DLD-1 cells exhibit a significant enhancement in hSMG1 (4.3±0.1) mRNA quantity in response to ouabain, the other members of the PIKK family display little or no change (ATR: 0.7±0.1-fold; ATM: 1.1±0.1-fold; DNAPK: 0.8±0.1-fold; mTOR: 1.3±0.1-fold).

The Na⁺/K⁺-ATPase is the common target of ouabain, digoxin, and digitoxin (Buckalew, 2005). Na⁺/K⁺-ATPase inhibition induces a plethora of effects including (a) disruption of the Na⁺/K⁺ gradient and consequent increase in intracellular [Ca²⁺] (Grapengiesser et al., 1993), (b) interference with amino acid uptake (Johnson et al., 1986), and (c) the production of reactive oxygen species resulting in oxidative damage to proteins and organelles (Xie et al., 1999). Each of these individual events is either sufficient or necessary to promote autophagy. The latter maintains cell viability by furnishing essential biochemical building blocks via degradation of existing proteins and organelles in lysosomes (Levine and Klionsky, 2004). However, the effect of ouabain, or its cardiotonic congeners, on autophagy has not been examined. The later was explored by measuring rates of degradation of long-lived proteins, the preferential substrate for lysosomal degradation, in DLD-1 cells. As is evident from FIG. 2a, ouabain dramatically enhances proteolysis and to an extent that is significantly greater than amino acid starvation alone. The ouabain-induced increase in protein degradation is completely blocked in the presence of ammonium chloride and leupeptin, general inhibitors of lysosomal-driven protein degradation (FIG. 2b). Furthermore, 3-methyladenine, a known inhibitor of macroautophagy (Cuervo, 2005), partially offsets the effect of ouabain as well (30.5±3.4% of lysosomal degradation is sensitive to 3-methyladenine) (FIG. 2c). The latter implies that macroautophagy, the degradation of sequestered material in a vesicular-based fashion, along with other types of autophagy (i.e. chaperone-mediated autophagy and microautophagy) contribute to ouabain-induced proteolysis.

Agents that block the ouabain-induced upregulation of hSMG1 expression were also identified by screening the drug library in the presence of ouabain (10 μM) (Table 2). It was anticipated that drugs identified in the counter-screen might target pathway components essential for modulating or relaying the signal initiated by Na⁺/K⁺-ATPase inhibition. Library members that suppress ouabain-induced hSMG1 expression include agents that block the release of Ca²⁺ into the intracellular environment (jasplakinolide, nifedipine, and 2-aminoethoxydiphenylborate) (Aizman et al., 2001). The latter is consistent with the notion that autophagy and hSMG1 expression levels respond to a shared environmental stimulus since autophagy is dependent upon Ca2+ release from intracellular stores (Gordon et al., 1993). Ouabain's action on hSMG1 expression is also blocked by apigenin (1.0±0.1 versus untreated cells), a flavonoid that inhibits drug-induced autophagy (Gordon et al., 1995). Several other compounds interfere with ouabain action, including a variety of caspase inhibitors, metal chelators, and inhibitors of nucleic acid synthesis (Table 2).

TABLE 2
Compounds that Block Ouabain-Induced hSMG1 Overexpression.

| | hSMG1 |
|---|---|
| Drug (concentration) | Expression |
| Ouabain (10 μM) | 4.3 ± 0.1 |
| Ouabain (10 μM) + Nifedipine (50 μM) | 1.5 ± 0.1 |
| Ouabain (10 μM) + Jasplakinolide (6 μM) | 0.85 ± 0.03 |
| Ouabain (10 μM) + aminoethoxydiphenylborate (500 μM) | 0.19 ± 0.04 |
| Ouabain (10 μM) + Apigenin (28 μM) | 1.0 ± 0.1 |
| Ouabain (10 μM) + Ciclopirox (28 μM) | 0.53 ± 0.02 |
| Ouabain (10 μM) + Hinokitiol (250 μM) | 1.3 ± 0.1 |
| Ouabain (10 μM) + Actinomycin D (6 μM) | 0.37 ± 0.01 |
| Ouabain (10 μM) + Mitoxantrone (14 μM) | 0.47 ± 0.01 |
| Ouabain (10 μM) + Z-DEVD-FMK (caspase 3 | 1.2 ± 0.4 |
| inhibitor; 50 μM) | |
| Ouabain (10 μM) + Z-VE(OMe)ID(OMe)- | 1.0 ± 0.1 |
| FMK (caspase 6 inhibitor; 50 μM) | |
| Ouabain (10 μM) + IETD-CHO (caspase 8 inhibitor; | 1.0 ± 0.1 |
| 50 μM) | |

DLD-1 cells were exposed to the library of FDA-approved drugs (7.5 μg/mL) and related agents in the presence of ouabain (7.5 μg/mL). Hits were re-examined in triplicate at the specified concentrations in the presence of 10 μM ouabain. hSMG1 expression in the presence of ouabain and drug is given relative to untreated cells.
Values are mean ± S.D. of three different wells.

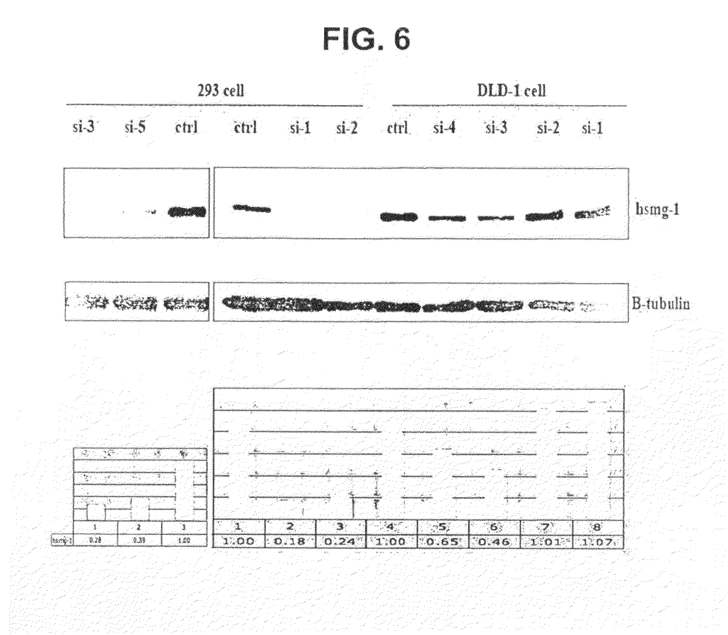

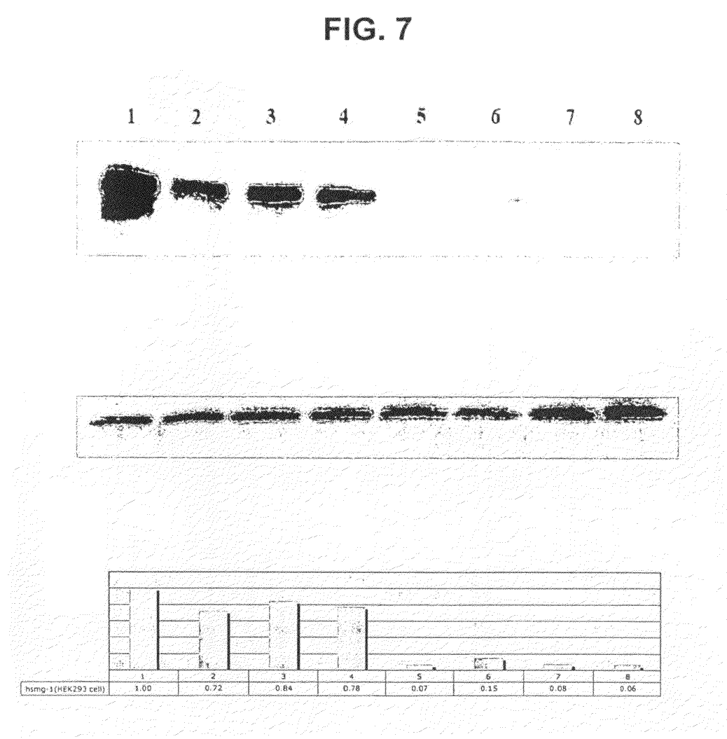

Autophagy is the quintessential cellular response to limited nutrient supplies and/or the presence of damaged cytoplasmic components (Levine and Yuan, 2005). The corresponding ouabain-induced overexpression of hSMG1 may be a biochemical mechanism by which metabolic homeostasis is re-established, either by inhibiting or promoting autophagy. The possible influence of hSMG1 on autophagy was examined via a series of RNAi knockdown experiments. Although an 80% decrease in hSMG1 protein levels was achieved in HEK293 cells, a more modest 50% decrease was observed in DLD-1 cells (FIG. 6). Consequently, all knockdown experiments were performed using the 293 cell line. Following RNAi knockdown, cells were either exposed to or deprived of amino acids and the effect on the rate of proteolysis noted. As a control, it was established that amino acid starvation alone does not significantly influence hSMG1 protein levels in the absence or presence of RNAi (FIG. 7). Protein degradation is enhanced in RNAi knockdown cells versus their unaltered counterparts (FIG. 3a). The combined influence of RNAi and amino acid starvation exacerbates the rate of degradation. Furthermore, the lysosomal inhibitor ammonium chloride only partly mitigates the effect of hSMG1 knockdown on protein degradation. This suggests that the RNAi-induced perturbation is mediated via both nonlysosomal and lysosomal-driven mechanisms (FIG. 3b).

A second classical cellular response to nutrient deprivation is the reduction in the rate of protein synthesis (Onodera and Ohsumi, 2005). Indeed, as is evident from FIG. 3c, protein synthesis (i.e. [³H]Leu incorporation) is sharply curtailed (43% of control) upon hSMG1 knockdown. Another member of the PIKK family, namely mTOR, has been implicated as the master regulator of protein synthesis and degradation (Raught et al., 2001). Specifically, mTOR inhibits autophagy and promotes transcription and translation (Meijer and Codogno, 2004; Arsham and Neufeld, 2006). These results suggest that hSMG1 can also participate as a regulator of global protein turnover (FIGS. 2 and 3). Furthermore, these results are consistent with and extend the consequences of the role of hSMG1 in mRNA splicing (Yamashita et al., 2005). Splicing enhances mRNA to protein translation rates in mammalian cells (Nott et al., 2004). Indeed, the nonsense-mediated decay effector up-frameshift 1, an hSMG1 substrate, has been demonstrated to markedly enhance translation (Nott et al., 2004). Consequently, it is reasonable that the loss of a key component of splicing (hSMG1) would slow the global rate of protein synthesis.

The strategy outlined in this study provides an expedient means to simultaneously assess the influence of a large array of biochemical sites of action on gene expression. Lead drugs identified from the screen furnish an immediate biological context since the mechanism of action of these agents is well understood as is the role played by their biochemical targets. As established here, hSMG1 is linked to the global rates of protein degradation (via both nonlysosomal and lysosomal-driven mechanisms) and protein synthesis, potentially placing it alongside mTOR as a key regulator of protein metabolism. Furthermore, the drugs identified in this study should be useful in directly assessing Abraham's conjecture that hSMG1 overexpression may biochemically compensate for the loss of ATM in patients with ataxia telangiectasia (Abraham, 2004). Finally, it was established that the steroid ouabain profoundly activates autophagy, a cellular self-preservation mechanism. It appears that ouabain is the first known example of a small, endogenously produced (adrenal glands) molecule that promotes autophagy.

### Materials and Methods

Drug Library Screen and hSMG1 message quantitation. The QuantiGene® Explore kit (Panomics, Inc.) was used to furnish a direct quantitation of mRNA in a 96 well format. The “housekeeper gene” 13-actin was selected as an intracellular control. Specific oligonucleotide probes for hSMG1, including capture extenders (CE), label extenders (LE) and blocking probes (BL) were designed with the Panomics ProbeDesigner™ software and synthesized. Prior to drug exposure, HeLa cells were incubated for 24 hr in 150 μL Dulbecco's modified Eagle Medium (DMEM) with 10% fetal bovine serum (FBS) and DLD-1 cells were incubated for 24 hr in 150 μL MEM with 1 mM MEM nonessential amino acids, 1 mM sodium pyrurate, 0.15% sodium bicarbonate, and 10% FBS. Cells were then exposed to various concentrations of the drugs for 24 hr and then lysed via incubation in 75 μL lysis buffer (QuantiGene® Explore kit) at 53° C. for 1 hr. Capture of hSMG1 and actin mRNA was conducted according to the manufacturers instructions and quantification performed using a microplate luminometer.

Fluorescence in situ hybridization (FISH). Previously described oligomer probes, each 50 bases in length from four regions of the hSMG1 gene, were designed with OLIGO software from Molecular Biology Insights, Inc., and synthesized on an Applied Biosystems automated DNA/RNA synthesizer (Capodieci et al., 2005). The activated fluorophore cy3 (Amersham) was used to label the probes. DLD-1 cells were seeded onto gelatinized coverslips, incubated as described above for 24 hr, and then treated with ouabain (10 μm) for 1 hr. After washing with PBSM (1×PBS with 5 mM MgCl₂), cells were extracted with PBST (1×PBS with 0.5% Triton x-100) for 1 min to remove the cytoplasm. The nuclei were subsequently fixed by exposing to PFA solution (4% paraformaldehyde in 1×PBS) for 20 min. The coverslips were placed into a pre-hybridization solution (50% formamide, 2×SSC) for at least 10 min. The coverslips were then removed, blotted, and placed cell side down, onto a plate containing one drop per coverslip of hybridization solution (RNA probes—20 ng per coverslip); and competitor (ssDNA/tRNA from Sigma, 100 fold excess to RNA probes) for 3 hr at 37° C. After washing with PBSM (37° C.), the coverslips were incubated in 2×SSC for 10 min. The nuclei were counter-stained with 4′,6-Diamidino-2-phenylindole and then washed with PBSM. Coverslips were mounted onto glass slides with the ProLong Antifade Kit (Molecular Probes). The FISH specimens were imaged and analyzed as previously described (Capodieci et al., 2005).

hSMG1 Protein Quantitation. DLD-1 cells were exposed to 10 μM ouabain for 24 hr and subsequently lysed. Total protein concentration, following centrifugation, was determined using the BCA Protein Assay Kit (Pierce). Following electrophoresis, the membrane was divided and the individual halves separately exposed to the SMG1 antibody BL1657 (Bethyl Laboratories) and the actin antibody A5441 (Sigma) and then subsequently incubated with the appropriate horseradish peroxidase conjugated secondary antibodies. The membranes were treated with the Pierce Supersignal West Pico chemiluminescent reagent and quantified.

Intracellular Protein Turnover. Rates of protein synthesis were measured in confluent cells as the incorporation of [3H]leucine [10 μCi/ml (1 Ci=37 GBq)] into acid-insoluble material in the presence of an excess (2.8 mM) of unlabeled leucine in the medium (to minimize differences due to alteration of amino acid transport and/or intracellular amino acid pool sizes). 28 To measure degradation of long-lived proteins, confluent cells were labeled with [3H]leucine (2 μCi/ml) for 48 h at 37° C. and then extensively washed and maintained in complete or amino acid depleted medium containing an excess of unlabeled leucine. Aliquots of the medium taken at different times were precipitated with trichloroacetic acid, and proteolysis was calculated as the amount of acid precipitable radioactivity converted into acid soluble at each time (Auteri et al., 1983). Where indicated, 20 mM NH₄Cl and 100 μM leupeptine or 10 mM 3-methyladenine were added during the chase to block lysosomal proteolysis or macroautophagy, respectively.

### REFERENCES

Abraham, R. T. DNA Repair (Amst.) 3, 883-887 (2004a).
Abraham, R. T. DNA Repair (Amst.) 3, 919-925 (2004b).
Aizman, O., Uhlen, P., Lal, M., Brismar, H. & Aperia, A. Proc Natl Acad Sci USA 98, 13420-13424 (2001).
Arsham, A. M., & Neufeld, T. P. Thinking globally and acting locally with TOR. Curr. Opin. Cell Biol. 18, 589-597 (2006).
Auteri, J. S., Okada, A., Bochaki, V. & Dice, J. F. J. Cell. Physiol. 115, 167-174 (1983).
Belz, G. G., Breithaupt-Grogler, K. & Osowski, U. Eur. J. Clin. Invest. 31 Suppl 2, 10-17 (2001).
Buckalew, V. M. Endogenous digitalis-like factors. An historical overview. Front. Biosci. 10, 2325-82334 (2005).
Bussolati, O. et al. Mol. Membr. Biol. 18, 27-38 (2001).
Capodieci, P., Donovan, M., Buchinsky, H., Jeffers, Y., Cordon-Cardo, C., Gerald, W., Edelson, J., Shenoy, S. M., & Singer, R. H. Nature Methods 2, 663-5 (2005). Collins, M. L. et al. Nucleic Acids Res. 25, 2979-2984 (1997).
Cuervo, A. M. Autophagy: Many paths to the same end. Mol. Cell. Biochem. 263, 55-72 (2005).
Cuervo A. M., Stefanis L., Fredenburg R., Lansbury P. T., & Sulzer D. Science 305, 1292-1295 (2004).
Gordon, P. B., Holen, I., Fosse, M., Rotnes, J. S. & Seglen, P. O. J. Biol. Chem. 268 26107-26112 (1993).
Gordon, P. B., Holen, I., & Seglen, P. O. Protection by naringin and some other flavonoids of hepatocytic autophagy and endocytosis against inhibition by okadaic acid. J. Biol. Chem. 270, 5830-5838 (1995).
Grapengiesser, E., Berts, A., Saha, S., Lund, P. E., Gylfe, E., & Hellman, B. Dual effects of Na/K pump inhibition on cytoplasmic Ca²⁺ oscillations in pancreatic beta-cells. Arch. Biochem. Biophys. 300, 372-377 (1993).
Johnson, M. D., Ash, J. F., & Pauw, P. G. Alterations in amino acid transport in Na,K-ATPase amplified HeLa cells. J. Biol. Chem. 261, 40-43 (1986).
Lee, S. J. et al. Br J Pharmacol 145, 469-476 (2005).
Levine B., & Klionsky D. J. Development by self-digestion: molecular mechanisms and biological functions of autophagy. Dev. Cell. 6, 463-77 (2004).
Levine, B. & Yuan, J. Autophagy in cell death: an innocent convict? J. Clin. Invest. 115, 2679-2688 (2005)
Mann, C. L., Bonner, C. D., Jewell, C. M. & Cidlowski, J. A. Endocrinology 142, 5059-5068 (2001).
Meijer, A. J., & Codogno, P. Regulation and role of autophagy in mammalian cells. Int. J. Biochm. Cell Biol. 36, 2445-2462 (2004).
Mendell, J. T., Sharifi, N. A., Meyers, J. L., Martinez-Murillo, F. & Dietz, H. C. Nat. Genet. 36, 1073-1078 (2004).
Niisato, N., Ito, Y. & Marunaka, Y. Biochem Biophys Res Commun 254, 368-371 (1999).
Nott, A., Le Hir, H., & Moore, M. J. How introns influence and enhance eukaryotic gene expression. Genes Dev. 18, 210-222 (2004).
Onodera J, Ohsumi Y. Autophagy is required for maintenance of amino acid levels and protein synthesis under nitrogen starvation. J. Biol. Chem. 280, 31582-31586 (2005).
Raught, B., Gingras, A. C., & Sonenberg, N. The target of rapamycin (TOR) proteins. Proc. Natl. Acad. Sci. USA 98, 7037-7044 (2001).
Root, D. E., Flaherty, S. P., Kelley, B. P., & Stockwell, B. R. Biological mechanism profiling using an annotated compound library. Chem. Biol. 10, 881-892 (2003).
Savchuk, N. P., Balakin, K. V., Tkachenko, & S. E. Exploring the chemogenomic knowledge space with annotated chemical libraries. Curr. Opin. Chem. Biol. 8, 412-417 (2004).
Scheiner-Bobis, G. & Schoner, W. Nat Med 7, 1288-1289 (2001).
Schoner, W. Eur. J. Biochem. 269, 2440-2448 (2002).
Therien, A. G. & Blostein, R. Am. J. Physiol. Cell Physiol. 279, C541-566 (2000).
Xi, B., Guan, F. & Lawrence, D. S. J Am Chem Soc 126, 5660-5661 (2004).
Xie, Z., Kometiani, P., Liu, J., Li, J., Shapiro, J. I., & Askari, A. Intracellular reactive oxygen species mediate the linkage of Na⁺/K⁺-ATPase to hypertrophy and its marker genes in cardiac myocytes. J. Biol. Chem. 274, 19323-19328 (1999).
Yamashita, A., Ohnishi, T., Kashima, I., Taya, Y. & Ohno, S. Genes Dev 15, 2215-2228 (2001).
Yamashita, A., Kashima, I., & Ohno, S. The role of SMG-1 in nonsense-mediated mRNA decay. Biochim. Biophys. Acta 1754, 305-315 (2005).

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method for identifying signaling pathways that regulate the expression of a test gene, the method comprising
(a) combining each of n cell cultures with each of n compounds, where a different compound is combined with each cell culture,
wherein the n cell cultures are capable of expressing the test gene, and wherein signaling pathways affected by each of the compounds are known;
(b) measuring expression of the test gene in each of the cell cultures to identify compounds (“active compounds”) that affect expression of the test gene in the cell culture;
(c) combining cell cultures with an active compound identified in step (b) and to at least a portion of the n compounds, where a different compound is combined with each cell culture;
(d) measuring expression of the test gene in each of the cell cultures of step (c) to identify compounds that negate the effect that the active compound has on expression of the test gene (“negating compounds”); and
(e) identifying the signaling pathways that are affected by a negating compound (“test gene pathways”), wherein the test gene pathways are signaling pathways that regulate the expression of the test gene.

2. The method of claim 1, wherein the test gene is a disease gene, an immunity gene, a cytokine gene, or a transcription factor gene.

3. The method of claim 1, wherein the test gene encodes an enzyme.

4. The method of claim 3, wherein the enzyme is a phosphatase or a kinase.

5. The method of claim 3, wherein the enzyme is a protein kinase.

6. The method of claim 5, wherein the protein kinase is a member of the phosphoinositide 3-kinase-related protein kinase (PIKK) family.

7. The method of claim 1, wherein the test gene encodes a signaling molecule.

8. The method of claim 1, wherein the cell is a prokaryote or a plant cell.

9. The method of claim 1, wherein the cell is a mammalian cell.

10. The method of claim 1, wherein the cell is a human cell.

11. The method of claim 10, wherein the cell is a HeLa cell.

12. The method of claim 1, wherein the cell naturally expresses the test gene.

13. The method of claim 1, wherein the cell is transfected with the test gene.

14. The method of claim 1, wherein the cell expresses an assayable peptide upon expression of the test gene.

15. The method of claim 14, wherein the assayable peptide is an antigen, an enzyme, or a fluorescent protein.

16. The method of claim 1, wherein expression of the test gene is measured by measuring test gene mRNA.

17. The method of claim 1, wherein expression of the test gene is measured by measuring the peptide encoded by the test gene.

18. The method of claim 1, wherein each of the n compounds is independently a protein, a lipid, a nucleic acid, a carbohydrate, or any combination thereof.

19. The method of claim 1, wherein each of the n compounds is a different organic compound less than about 2000 Da.

20. The method of claim 19, wherein each of the organic compounds is an FDA-approved drug.

21. The method of claim 1, wherein parts (c)-(e) are performed with a second active compound

22. The method of claim 1, wherein parts (c)-(e) are performed with each active compound.
